(19) United States
(12) Patent Application Publication
(10) Pub. No.: US 2008/0045930 A1
(43) Pub. Date: Feb. 21, 2008

(54) System and Method of Drug Identification Through Radio Frequency Identification (RFID)

(76) Inventors: Ronald Makin, Fitchburg, WI (US); Kyle Jansson, Blaine, MN (US); Silas Zirn, Madison, WI (US); David Andrzejewski, Madison, WI (US); Timothy Flink, Mahtolmedi, MN (US)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/465,993

(22) Filed: Aug. 21, 2006

(51) Int. Cl. A61K 9/22

(52) U.S. Cl. 604/890.1; 604/93.01

(57) ABSTRACT

The present invention is a system and method of drug identification through radio frequency identification “RFID.” The system and method is implemented where drugs are being administered, such as in a hospital or clinic, and identifies the drug being administered to the patient through the use of RFID tags and short and long range transceivers before the drug reaches the patient. After identification, the system and method is configured to cross reference the identified drug with the patient's prescription and allergy information, and to prevent delivery to the patient, if necessary.

### FIELD OF THE INVENTION

The invention relates to the field of drug delivery. More particularly, the invention relates to the field of identifying drug dosages before administering them to a patient.

### BACKGROUND OF THE INVENTION

Inappropriate administration of injected medication via syringe and IV tubing is a well-documented and serious problem. Administering a completely wrong drug can arise from syringe swapping or mislabeling. Inappropriate drug administration can also arise from a clinician giving a drug to address one problem or to invoke a response while being unaware of a potentially serious contraindication. For instance, administering a bolus of morphine to relieve pain in the patient whose cardiovascular condition is already compromised can be fatal.

Several reports suggest that many lives are lost due to preventable medication errors. The number of adverse drug events (ADE'S) suggest a system and/or method is required to ensure that the right drug and the right concentration is given at the right time to the right patient. The studies looked at where the errors actually occur and deduced that the majority occur at the point of delivery.

A November 1999 institute of medicine report states that “the medication process provides an example where implementing better systems will yield better human performance. Medication errors now occur frequently at hospitals, yet many hospitals are not making use of known safety systems, nor are they actively pursuing new safety systems.”

### SUMMARY OF THE INVENTION

The present invention is a system and method of drug identification through radio frequency identification “RFID.” The system and method is implemented where drugs are being administered, such as in a hospital or clinic, and identifies the drug being administered to the patient through the use of RFID tags and short and long range transceivers before the drug reaches the patient. After identification, the system and method is configured to cross reference the identified drug with the patient's prescription and allergy information, and to prevent delivery to the patient, if necessary.

One aspect of the present invention is a system for identifying a drug being administered to a patient, the system comprises a drug source, wherein the drug source includes the drug to be administered to the patient, a first radio frequency identification (RFID) tag affixed to the drug source, the first RIFD tag configured to emit a first radio frequency identifying the drug source, a second RFID tag affixed to the patient, the second RFID tag configured to emit a second radio frequency identifying the patient, a drug pump configured to dispense the drug from the drug source, an active tag in close proximity to the first and second RFID tags, the active tag configured to receive the first and second frequencies, and further configured to emit a third radio frequency corresponding to the received radio frequencies, a long range transceiver configured in a treatment area, and further configured to receive the third radio frequency and a processing means coupled to the long range transceiver, wherein the processing means processes the third radio frequency and confirms that the drug is appropriate for the patient, and further records the drug administration. The processing means of this system cross-references the third radio frequency with a set of databases and is configured to trigger an alarm when the drug is not appropriate for the patient. The processing means is configured to activate a drug stop mechanism when the drug is not appropriate for the patient and the active tag is affixed to the drug pump. The drug source of the system may be a syringe and the drug pump is a syringe pump. The drug source may also be a drug bag, and further wherein the first RFID tag is affixed to a line running from the drug bag to the drug pump which may be a volumetric pump. The system further comprises a third RFID tag fixed to a drug administrator, the third RFID tag configured to emit a fourth radio frequency identifying the drug administrator, such that the drug administrator may be recorded by the processing means. The drug source of this system is a prepared medication, and the treatment area is a pharmacy, and further wherein the drug pump is not utilized and the active tag is configured in close proximity to the prepared medication and wherein the active tag is a short range transceiver.

Another aspect of the present invention is a method of identifying a drug being administered to a patient, the method comprises affixing an active tag to a drug pump, wherein the drug pump is configured to dispense the drug being administered from a drug source to the patient, configuring a long range transceiver in a treatment area, affixing a radio frequency (RFID) tag to the drug source, the patient and an administering personnel, wherein the RFID tag is configured to emit a first radio frequency identifying each of the drug source, the patient and the administering personnel, reading the first radio frequency with the active tag, wherein the active tag is configured to emit a second radio frequency corresponding to the first radio frequency, reading the second radio frequency with the long range transceiver and processing the second radio frequency, wherein the processing step includes confirming and recording the drug administration. The confirming step of the method includes cross-referencing the second radio frequency with a set of databases and further comprises triggering an alarm when the drug is not appropriate for the patient triggering a drug stop mechanism when the drug is not appropriate for the patent. The drug source of the method maybe a syringe and the drug pump is a syringe pump. The drug source may also be a drug bag and the drug pump may also be a volumetric pump and further wherein the RFID tag is affixed to a line running from the drug bag to the drug pump. The drug source of the method may be a prepared medication, and the treatment area is a pharmacy, and further wherein the drug pump is not utilized and the active tag is configured in close proximity to the prepared medication and the active tag may be a short range transceiver.

### BRIEF DESCRIPTION OF THE DRAWINGS

FIG. 1 is a graphical representation illustrating an embodiment of the system of the present invention.

FIG. 2 is a block diagram illustrating an embodiment of the system of the present invention.

FIG. 3 is a graphical representation illustrating an embodiment of the system of the present invention.

FIG. 4 is a flow chart illustrating an embodiment of the method of the present invention.

### DETAILED DESCRIPTION

Drugs are given to patients by many routes: intravenously; epidurally; orally; etc. The present invention currently applies to drugs given by infusion devices into the vein or other fluid routes into the body. Infusion devices are generally classified into two main categories: syringe pumps and volumetric pumps. Patients in the operating room and ICU are often being treated with a multitude of drugs. The present invention includes a method and system for detecting which drug is in which particular pump.

It is also contemplated that the system described could detect the attending clinician and the patient. In detecting the patient undergoing treatment, the system could also advise if there are particular contra-indications of the drugs for that particular patient. Although pharmacy dispensing errors are thought to be less of an issue, the present invention could be used as a verification and validation method.

The system and method utilizes a combination of long range and short range transceivers along with radio frequency identification (RFID) tags. The long range transceiver preferably covers a distance of about 10 meters, giving it the ability to scan an operating room, ICU area, or other hospital treatment area. The short range transceivers act as the communication between the tagged drug dispenser, syringe or fluid bag, and the long range transceiver, thereby associating a particular drug to a particular infusion device.

Preferably, the system can be organized such that it accomplishes the goal of ensuring the right drug is administered to the right patient in the right concentration at the right time. When connected to a computer, the system can also be used in conjunction with medication and patient data to ensure the patient does not receive any drugs that are contra-indicated, for example, allergies. The computer can also ensure that the particular drug is given within its therapeutic range to prevent drug over/under doses due to incorrect data entry errors.

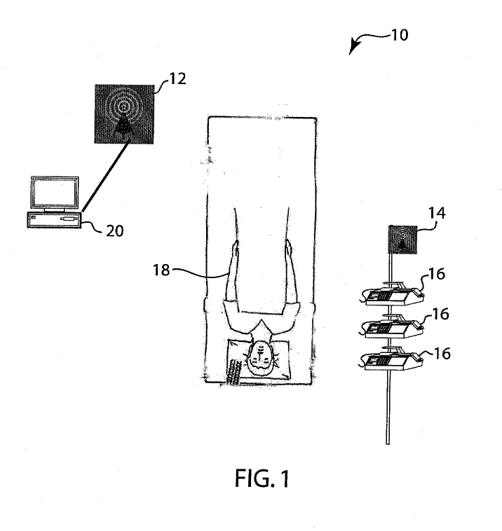

FIG. 1 shows the drug ID system 10 employed for a multitude of syringe pumps 16. In this example, three pumps 16 are shown. The long range transceiver 12 scans the whole area and detects the short range transceiver 14 associated with each pump 16.

Still referring to FIG. 1, the drug ID system 10 includes a long range transceiver 12, a processing means such as a system workstation 20, at least one syringe pump 16, and a short range transceiver, also referred to as an active tag, all being used to identify a drug being administered to a patient 18. In this embodiment, three syringe pumps 16 are utilized to deliver three different drugs to the patient 18. It should be noted that this system 10 may utilize multiple syringe pumps 16, corresponding to the number of drugs being administered to the patient 18, and as few as one syringe pump 16 when only one drug is being administered. The short range transceiver 14, or active tag, may be configured in close proximity to the syringe pumps 16, or multiple short range transceivers 14 may be configured in close proximity to each of the syringe pump 16, or preferably be affixed to each syringe pump 16. Syringe pumps 16 are operated by inserting a syringe containing the drug to be administered into the syringe pump 16, and programming the syringe pump 16 to administer the appropriate dosage to the patient 18. In a preferred embodiment, a radio frequency identification (RFID) tag would be attached to each syringe prior to inserting it into the syringe pump 16. In that way, the syringe pumps 16 may still be utilized for a variety of different drugs. In such an embodiment, the RFID tag would be read by the short range transceiver 14, and the short range transceiver 14 would emit a corresponding signal.

Still referring to FIG. 1, the drug ID system 10 will also include a long-range transceiver 12. The long range transceiver 12 will be configured in a treatment area such as an operating room, ICU area, or other hospital treatment area, and would be configured to receive signals emitted by any and all short range transceivers 14 in that treatment area. The information collected by the long range transceiver 12 would be inputted into a processing means such as a system workstation 20. The system workstation 20 is preferably configured to cross-reference the drug information with a set of databases. Such set of databases may include patient medical history databases, current patient pharmacy databases, conflicting drug databases, as well as any other databases required to make sure that the drug being administered to the patient 18 is the correct drug, and is not harmful to the patient. The system workstation 20, upon finding that a drug being administered is dangerous or inappropriate, may also be configured to sound an alarm, or even trigger a drug stop mechanism on the appropriate syringe pump 16 as well.

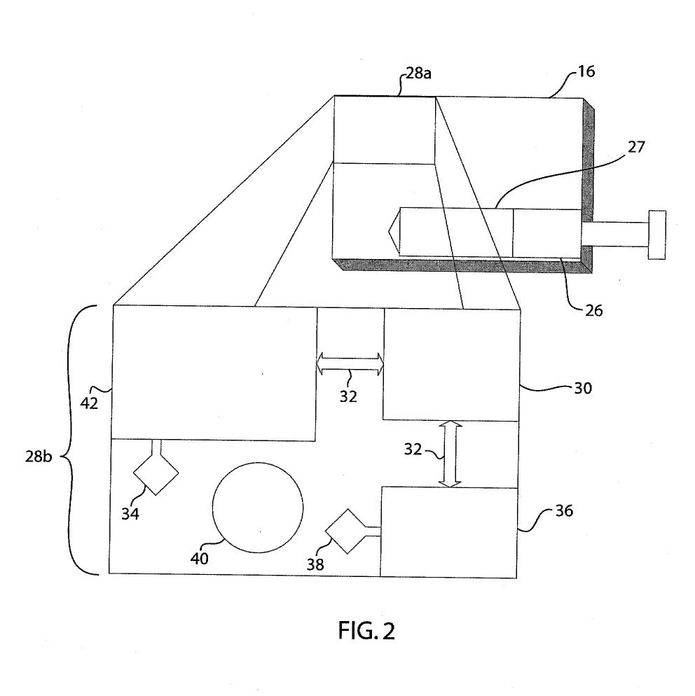

FIG. 2 shows more detail of the active tag 28 attached to the individual pumps 16. This active tag 28 obtains the information contained in the RFID tag 26 attached to the syringe 16 and transmits this to the host transceiver 12 (not shown). In this way, that particular tagged syringe is associated with that particular pump 16. The communication port of the pump 16 attached to the computer and/or controller identifies the pump 16 by means of a serial number or other pump identifying means. The patient 18 has an RFID tag 26 such that the medication system is associated with that patient 18, which enables the analysis to prevent contra-indicated drugs as mentioned above, or prevent the drug from being administered at that particular time.

Still referring to FIG. 2, an exemplary syringe pump 16 as depicted in FIG. 1, is shown in more detail. Here, the syringe 27 is inserted into the syringe pump 16, including an RFID tag 26 affixed to the syringe 27. In this embodiment, the short range transceiver or active tag 28 is affixed directly to the syringe pump 16. The magnified active tag 28b, includes a block diagram of the components of the active tag 28a. This view shows a short range antenna 38 and short range reader 36 configured to read the RFID tag 26 on the syringe 27. A data communication interface 32 relays this information to the microcontroller 30 which utilizes another communication interface 32 in order to relay the signal to the long range transmitter 42. The long range transmitter 42 utilizes a long range antenna 34 to transmit this information, eventually to a long range transceiver 12 (not shown). The active tag 28 is powered by a battery 40.

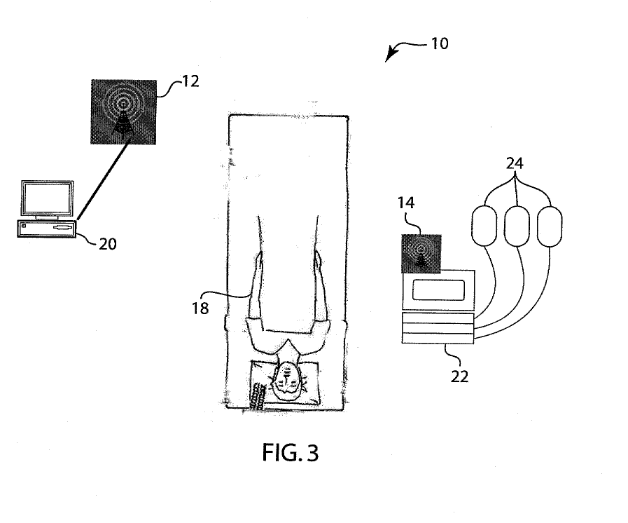

FIG. 3 shows the drug ID system 10 employed for a multitude of drugs and fluids being delivered by volumetric pumps 22. Volumetric pumps 22 are either individual devices, or as shown in this instance, a central control unit with several channels of pumping mechanisms. Volumetric pump 22 drug delivery is more difficult in identifying which channel or device is delivering which drug because of the remote positioning of the drugs in the bags 24. In this instance, the bag 24 is tagged prior to connection of the lines. Once the line is connected to the bag 24, the tag on the bag 24 will be moved closer to the pumping mechanism, and hence the ability to detect which device or channel is delivering which drug by association in a similar manner to the FIG. 2.

Referring again to FIG. 3, the drug ID system 10 of the present invention may also include a volumetric pump 22. As was shown in FIG. 1, the drug ID system 10 in FIG. 3 will also utilize a long range transceiver 12, a system workstation 20, and a short range transceiver or active tag 14 in order to identify a drug being administered to a patient 18. However, in this embodiment, a number of drug bags 24 will be utilized in conjunction with a volumetric pump 22 in order to deliver the drugs to the patient 18. In such a case, RFID tags corresponding to each drug in each drug bag 24 will be attached to the line from the drug bag 24 to the volumetric pump 22. In this way, a short range transceiver 14 may still be attached to the volumetric pump 22, and still be in range to read the RFID tags. An alternative embodiment will include a plurality of volumetric pumps 22 for each of the drug bags 24 administering drugs to the patient 18. In such embodiments, there may be multiple short range transceivers 14, or a single short range transceiver 14 able to read each of the RFID tags corresponding to the drug bags 24. It should be noted that the short range transceivers are configured to read RFID tags in a range from approximately 0-3 centimeters. Also, while it is stated that the preferable range for the long range transceiver is 10 meters, the long range transceiver may read the short range transceiver 14 in a range from 0 to 15 meters.

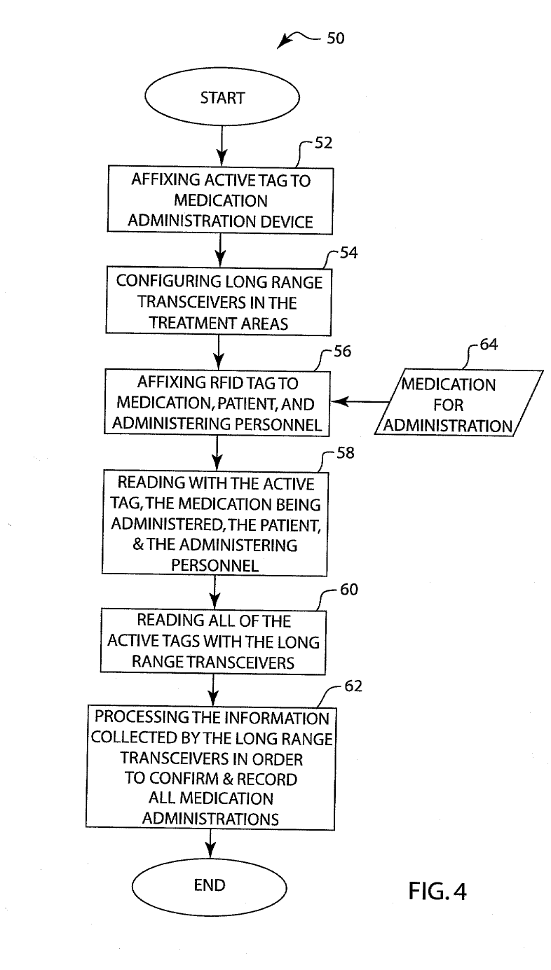

Referring now to FIG. 4, a drug ID method 50 of the present invention is depicted. In step 52, an active tag is affixed to a medication administration device such as a syringe pump or a volumetric pump. In step 54, a long range transceiver is configured in the desired treatment areas. In step 56, a medication for administration is tagged with an RFID tag, and an RFID tag is affixed to the patient and the administering personnel as well. In step 58, the active tag reads the medication being administered, the patient and the administering personnel information. In step 60, the long range transceivers read all of the active tags, and in step 62 the information collected by the long range transceiver is processed in order to confirm the medication and record all medication administrations for the hospital or clinic records.

It should also be noted that this invention is not limited solely to syringe pumps and volumetric pumps, as any known method of administering drugs to a patient may be utilized and further monitored using the RFID tags and active tag method and system. It should also be noted that any administering personnel in a hospital or clinic may wear RFID tags such that their information can be read by an active tag and subsequently read by the long range transceiver and recorded by the processing means. Lastly, it is also contemplated that such a system may be implemented in a pharmacy setting, wherein a single long range transceiver is positioned in the pharmacy and each prepared medication is given an RFID tag. Numerous active tags may be placed throughout the pharmacy in order to continuously record all medication leaving the pharmacy, recording the individual sending them out of the pharmacy, and the patient receiving them from the pharmacy.

The overall scheme of the present invention is to be able to associate which drug is being delivered by which infusion apparatus, and ultimately to a particular patient. The present invention may also be configured to detect the attending clinician who sets the delivery of the drugs. In this way, the system will insure that incorrect drug delivery is reduced and that adverse drug events are minimized. In a system that has central control and/or display, it will also insure that the control or display is aware of which drug or fluid is in which infusion apparatus.

The system and method are advantages over the prior art in that it will help insure the right drug in the right concentration is delivered to the right patient at the right time, and further associates which delivery device has which drug such that remote control and/or display is possible, thus preventing adverse drug events. A pharmacy application may also be configured to validate the drugs being dispensed.

The present invention has been described in terms of specific embodiments incorporating details to facilitate the understanding of the principals of construction and operation of the invention. Such reference herein to specific embodiments and details thereof is not intended to limit the scope of the claims appended hereto. It will be apparent to those skilled in the art that modifications may be made in the embodiment chosen for illustration without departing from the spirit and scope of the invention.

What is claimed is:

1. A system for identifying a drug being administered to a patient, the system comprising:
a drug source, wherein the drug source includes the drug to be administered to the patient;
a first radio frequency identification (RFID) tag affixed to the drug source, the first RIFD tag configured to emit a first radio frequency identifying the drug source;
a second RFID tag affixed to the patient, the second RFID tag configured to emit a second radio frequency identifying the patient;
a drug pump configured to dispense the drug from the drug source;
an active tag in close proximity to the first and second RFID tags, the active tag configured to receive the first and second frequencies, and further configured to emit a third radio frequency corresponding to the received radio frequencies;
a long range transceiver configured in a treatment area, and further configured to receive the third radio frequency; and
a processing means coupled to the long range transceiver, wherein the processing means processes the third radio frequency and confirms that the drug is appropriate for the patient, and further records the drug administration.

2. The system as claimed in claim 1, wherein the processing means cross-references the third radio frequency with a set of databases.

3. The system as claimed in claim 1, wherein the processing means is configured to trigger an alarm when the drug is not appropriate for the patient.

4. The system as claimed in claim 1, wherein the processing means is configured to activate a drug stop mechanism when the drug is not appropriate for the patient.

5. The system as claimed in claim 1, wherein the active tag is affixed to the drug pump.

6. The system as claimed in claim 1, wherein the drug source is a syringe.

7. The system as claimed in claim 6, wherein the drug pump is a syringe pump.

8. The system as claimed in claim 1, wherein the drug source is a drug bag, and further wherein the first RFID tag is affixed to a line running from the drug bag to the drug pump.

9. The system as claimed in claim 8, wherein the drug pump is a volumetric pump.

10. The system as claimed in claim 1, further comprising a third RFID tag fixed to a drug administrator, the third RFID tag configured to emit a fourth radio frequency identifying the drug administrator, such that the drug administrator may be recorded by the processing means.

11. The system as claimed in claim 1, wherein the drug source is a prepared medication, and the treatment area is a pharmacy, and further wherein the drug pump is not utilized and the active tag is configured in close proximity to the prepared medication.

12. The system as claimed in claim 1, wherein the active tag is a short range transceiver.

13. A method of identifying a drug being administered to a patient, the method comprising:
affixing an active tag to a drug pump, wherein the drug pump is configured to dispense the drug being administered from a drug source to the patient;
configuring a long range transceiver in a treatment area;
affixing a radio frequency (RFID) tag to the drug source, the patient and an administering personnel, wherein the RFID tag is configured to emit a first radio frequency identifying each of the drug source, the patient and the administering personnel;
reading the first radio frequency with the active tag, wherein the active tag is configured to emit a second radio frequency corresponding to the first radio frequency;
reading the second radio frequency with the long range transceiver; and
processing the second radio frequency, wherein the processing step includes confirming and recording the drug administration.

14. The method as claimed in claim 13, wherein the confirming step includes cross-referencing the second radio frequency with a set of databases.

15. The method as claimed in claim 13, further comprising triggering an alarm when the drug is not appropriate for the patient.

16. The method as claimed in claim 13, further comprising triggering a drug stop mechanism when the drug is not appropriate for the patent.

17. The method as claimed in claim 13, wherein the drug source is a syringe.

18. The method as claimed in claim 17, wherein the drug pump is a syringe pump.

19. The method as claimed in claim 13, wherein the drug source is a drug bag, and further wherein the RFID tag is affixed to a line running from the drug bag to the drug pump.

20. The method as claimed in claim 19, wherein the drug pump is a volumetric pump.

21. The method as claimed in claim 13, wherein the drug source is a prepared medication, and the treatment area is a pharmacy, and further wherein the drug pump is not utilized and the active tag is configured in close proximity to the prepared medication.

22. The method as claimed in claim 13, wherein the active tag is a short range transceiver.

23. A drug identification system, the system comprising:
a drug source, wherein the drug source includes a drug to be administered to a patient;
a first radio frequency identification (RFID) tag affixed to the drug source, the first RIFD tag configured to emit a first radio frequency identifying the drug source;
a second RFID tag affixed to the patient, the second RFID tag configured to emit a second radio frequency identifying the patient;
a third RFID tag affixed to a drug administrator, the third RFID tag configured to emit a third radio frequency identifying the drug administrator;
a drug pump configured to dispense the drug from the drug source;
a short range transceiver in close proximity to the RFID tags, the short range transceiver configured to receive the radio frequencies, and further configured to emit a fourth radio frequency corresponding to the received radio frequencies;
a long range transceiver configured in a treatment area, and further configured to receive the fourth radio frequency; and
a processing means coupled to the long range transceiver, wherein the processing means cross-references the fourth radio frequency with a set of databases to confirm that the drug is appropriate for the patient, and further records the drug administration.
